(19) United States
(12) Patent Application Publication
(10) Pub. No.: US 2023/0248678 A1
(43) Pub. Date: Aug. 10, 2023

(54) GEMCABENE FOR TREATING INFLAMMATION

(71) Applicant: NeuroBo Pharmaceulicals, Inc., Boston, MA (US)

(72) Inventors: Richard KANG, Boston, MA (US); Chares L. BISGAIER, Ann Arbor, MI (US)

(21) Appl. No.: 18/003,290

(22) PCT Filed: Jun. 24, 2021
(86) PCT No.: PCT/US2021/038917

§ 371 Date: Dec. 23, 2022

Related U.S. Application Data
(60) Provisional application No. 63/043,261, filed on Jun. 24, 2020.

(51) Int. Cl. A61K 31/194; A61P 31/14; A61P 37/06; A61P 11/00

(52) U.S. Cl. CPC A61K 31/194; A61P 31/14; A61P 37/06; A61P 11/00

(57) ABSTRACT

The present disclosure provides methods of treating and/or preventing cytokine storms with gemcabene or a pharmaceutically acceeptable salt thereof. Also disclosed are methods of treating inflammation caused by viral infections.

### CROSS-REFERENCE TO RELATED APPLICATIONS

This application claims priority to U.S. Provisional Application No. 63/043,261, filed Jun. 24, 2020, which is incorporated herein by reference in its entirety.

### FIELD

The present disclosure provides methods of treating and/or preventing cytokine storms, with gemcabene, or a pharmaceutically acceeptable salt thereof. Also disclosed are methods of treating inflammation caused by infections, e.g., viral infections.

### BACKGROUND

Influenza is the most common respiratory illness and, while generally seasonal, spreads rapidly and globally, affecting all age groups. Seasonal influenza accounts for greater than 200,000 hospitalizations and greater than 30,000 deaths per year in the United States. More recently, a previously unknown respiratory illnesss, SARS-CoV-2 quickly reached pandemic status and as of Jun. 15, 2020, has caused more than 434,000 deaths worldwide. In many cases, patients afflicted with these and other respiratory illnesses develop acute respiratory distress syndrome (ARDS) and multiple-organ failure as a result of a hyperactive immune reaction that releases excessive Interleukin-6 (IL-6), causing a cytokine rampage through the bloodstream. Known as “cytokine release syndrome” or a “cytokine storm,” this reaction triggers a severe inflammatory response that causes these conditions to develop.

Vaccination is the most effective manner to control respiratory virus-induced outbreaks and avoid complications which result from infection. However, in view of the difficulty to prepare, distribute and immunize the world population against an epidemic or pandemic, there is a clear and apparent need to develop more efficient methods to reduce the morbidity and mortality of respiratory viruses by preventing or treating cytokine storm responses in infected patients.

### SUMMARY

In certain aspects, the present disclosure provides a method of treating and/or preventing a cytokine storm in a subject in need thereof, the method comprising administering to the subject a pharmaceutical composition comprising an effective amount of gemcabene, or a pharmaceutically acceptable salt thereof. In some aspects, the cytokine storm is caused by or derived from a viral infection, a bacterial infection, a protozoan infection, a fungal infection, a prion, a vaccine, a vaccine booster, a gene therapeutic agent, a monoclonal antibody, or an allergan, or a combination thereof. In some aspects, the cytokine storm is caused by or derived from a viral infection. In certain aspects, the cytokine storm is caused by or derived from a viral infection which is a respiratory infection, a gastrointestinal tract infection, or a combination thereof. In some aspects, the viral infection is caused by an RNA or DNA virus. In some aspects, the virus has a filamentous, isometric, enveloped, or a head and tail conformation. In certain aspects, the virus is a coronavirus. In some aspects, the coronavirus is HCoV 229E, HCoV OC43, HCoV NL63, HCoV HKU1, SARS CoV, MERS CoV, SARS CoV-2, or a mutated strain thereof, or any combination thereof. In certain aspects, the coronavirus is SARS CoV-2. In some aspects, the coronavirus is a mutated strain of SARS CoV-2.

In certain aspects, the effective amount of gemcabene, or the pharmaceutically acceptable salt thereof, is from about 25 mg/day to about 900 mg/day. In some aspects, the effective amount of gemcabene is about 25 mg/day, about 50 mg/day, about 75 mg/day, about 100 mg/day, about 150 mg/day, about 200 mg/day, about 300 mg/day, about 400 mg/day, about 450 mg/day, about 500 mg/day, about 600 mg/day, about 700 mg/day, about 750 mg/day, about 800 mg/day, or about 900 mg/day.

In certain aspects, the gemcabene, or the pharmaceutically acceptable salt thereof, is administered for about 7 to about 28 days. In some aspects, the gemcabene, or the pharmaceutically acceptable salt thereof, is administered for about 7 to about 14 days. In some aspects, the gemcabene, or the pharmaceutically acceptable salt thereof, is administered for about 7, about 8, about 9, about 10, about 11, about 12, about 13, about 14, about 15, about 16, about 17, about 18, about 19, about 20, or about 21 days.

In certain asspects the pharmaceutical composition comprises the monocalcium salt of gemcabene.

In some aspects, the present disclosure provides a method of treating an inflammatory lung condition or disease caused by a viral infection in a subject in need thereof, the method comprising administering to the subject a pharmaceutical composition comprising an effective amount of gemcabene, or a pharmaceutically acceptable salt thereof. In certain aspects, the inflammatory lung condition or disease comprises an acute inflammation of the lung and/or an interstitial lung disease. In some aspects, the viral infection comprises an infection by a coronavirus, an influenza virus, and/or HIV. In certain aspects, the viral infection comprises a coronavirus. In some aspects, the viral infection comprises a coronavirus, wherein the coronavirus is HCoV 229E, HCoV OC43, HCoV NL63, HCoV HKU1, SARS CoV, MERS CoV, SARS CoV-2, or a mutated strain thereof, or any combination thereof. In certain aspects the coronavirus is SARS CoV-2. In some aspects, the coronavirus is a mutated strain of SARS CoV-2.

In certain aspects, the effective amount of gemcabene, or the pharmaceutically acceptable salt thereof, is from about 25 mg/day to about 900 mg/day. In some aspects, the effective amount of gemcabene, or the pharmaceutically acceptable salt thereof, is about 25 mg/day, about 50 mg/day, about 75 mg/day, about 100 mg/day, about 150 mg/day, about 200 mg/day, about 300 mg/day, about 400 mg/day, about 450 mg/day, about 500 mg/day, about 600 mg/day, about 700 mg/day, about 750 mg/day, about 800 mg/day, or about 900 mg/day.

In certain aspects, the gemcabene, or the pharmaceutically acceptable salt thereof, is administered for about 7 to about 28 days. In some aspects, the gemcabene, or the pharmaceutically acceptable salt thereof, is administered for about 7 to about 14 days. In certain aspects, the gemcabene, or the pharmaceutically acceptable salt thereof, is administered for about 7, about 8, about 9, about 10, about 11, about 12, about 13, about 14, about 15, about 16, about 17, about 18, about 19, about 20, or about 21 days.

In some aspects, the pharmaceutical composition comprises the monocalcium salt of gemcabene.

In certain aspects, the inflammatory lung condition or disease caused by the viral infection is hypercytokinaemia, haemophagocytic lymphohistiocytosis, pneumonia, acute respiratory distress syndrome, or systemic inflammatory response syndrome. In some aspects, the inflammatory lung condition or disease caused by the viral infection is acute respiratory distress syndrome.

In certain aspects, the subject has a metabolic disease. In some aspects, the subject has type 1 diabetes, type 2 diabetes, gestational diabetes, familial hypercholesterolemia, elevated triglycerides, above normal BMI, a BMI >40, cystic fibrosis, a history of smoking, idiopathic pulmonary fibrosis, idiopathic respiratory distress syndrome, non-alcoholic fatty liver disease (NAFLD), non-alcoholic steatohepatitis (NASH), liver disease, cirrhosis, hypertension, familial partial lipodystrophy, compromised respiratory function, asthma, chronic lung disease, chronic obstructive pulmonary disease (COPD), emphysema, chronic bronchitis, chronic kidney disease, a hemoglobin disorder, sickle cell anemia, thalassemia, anemia, a compromised immune system from cancer treatment, an autoimmune disease, an age of 65 or older, cardiovascular disease, a heart condition, heart failure, coronary artery disease, congenital heart disease, cardiomyopathies, pulmonary hypertension, vitamin D deficiency, or a combination thereof.

In certain aspects, the present disclosure provides a method of treating a multisystem inflammatory syndrome in a subject in need thereof, the method comprising administering to the subject a pharmaceutical composition comprising an effective amount of gemcabene, or a pharmaceutically acceptable salt thereof. In some aspects, the syndrome comprises fever that lasts more than 5 days and gets higher, severe abdominal pain, vomiting, or diarrhea, bloodshot eyes, skin rash, change in skin color, which can include becoming pale, patchy, or blue, difficulty feeding or too sick to drink, trouble breathing or quick breathing, chest pain or racing heart, confusion, irritability, or lethargy, or any combination thereof. In some aspects, the syndrome further comprises swelling and redness in the hands and feet, redness or cracking in the lips or tongue, swollen lymph nodes in the neck, or any combination thereof. In certain aspects, the syndrome is associated with a viral infection. In some aspects, the viral infection comprises an infection by a coronavirus, an influenza virus, and/or HIV. In certain aspects, the viral infection comprises an infection by a coronavirus wherein the coronavirus is HCoV 229E, HCoV OC43, HCoV NL63, HCoV HKU1, SARS CoV, MERS CoV, SARS CoV-2, a mutated strain thereof, or any combination thereof. In some aspects coronavirus is SARS CoV-2. In some aspects the coronavirus is a mutated strain of SARS CoV-2.

In certain aspects, the effective amount of gemcabene, or the pharmaceutically acceptable salt thereof, is from about 25 mg/day to about 900 mg/day. In some aspects, the effective amount of gemcabene, or the pharmaceutically acceptable salt thereof, is about 25 mg/day, about 50 mg/day, about 75 mg, /day about 100 mg/day, about 150 mg/day, about 200 mg/day, about 300 mg/day, about 400 mg/day, about 450 mg/day, about 500 mg/day, about 600 mg/day, about 700 mg/day, about 750 mg/day, about 800 mg/day, or about 900 mg/day.

In some aspects, the gemcabene, or the pharmaceutically acceptable salt thereof, is administered for about 7 to about 28 days. In certain aspects, the gemcabene, or the pharmaceutically acceeptable salt thereof, is administered for about 7 to about 14 days. In some aspects, the gemcabene, or the pharmaceutically acceeptable salt thereof, is administered for about 7, about 8, about 9, about 10, about 11, about 12, about 13, about 14, about 15, about 16, about 17, about 18, about 19, about 20, or about 21 days.

In certain aspects the pharmaceutical composition comprises gemcabene calcium.

In some aspects, the method further comprises administering one or more optional therapeutic agents to the subject.

In certain aspects, the subject is a pediatric. In some aspects, the subject is less than 18 years old, less than 30 years old, less than 50 years old, less than 60 years old, less than 75 years old, less than 80 years old, less than 85 years old, less than 90 years old, less than 95 years old, and/or less than 100 years old.

In certain aspects the present disclosure provides a kit comprising gemcabene, or a pharmaceutically acceptable salt thereof, for administering gemcabene, or a pharmaceutically acceptable salt thereof, to a subject having an inflammatory lung condition or disease caused by a viral infection, acute inflammation of the lung, or idiopathic pulmonary fibrosis.

### DETAILED DESCRIPTION

The present disclosure provides methods of treating and/or preventing cytokine storms with gemcabene, or a pharmaceutically acceeptable salt thereof,. Also disclosed are methods of treating inflammation caused by infections, e.g., viral infections, with gemcabene, or a pharmaceutically acceeptable salt thereof.

### I. Definitions

In order that the present description can be more readily understood, certain terms are first defined. Additional definitions are set forth throughout the detailed description.

It is to be noted that the term “a” or “an” entity refers to one or more of that entity.

As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “about” is used herein to mean approximately, roughly, around, or in the regions of When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The terms “administration,” “administering,” and grammatical variants thereof refer to introducing a composition, of the present disclosure, into a subject via a pharmaceutically acceptable route. The introduction of a composition of the present disclosure, into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject” and “patient” are used interchangeably herein. The human can be a male or female, child, adolescent, or adult. The female can be premenarcheal or postmenarcheal. In some aspects, the subject is an adult.

The term “therapeutically effective amount” or “therapeutically effective dosage” refers to an amount of an agent (e.g., gemcabene) that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. An effective amount can be administered in one or more administrations.

In some aspects, a “therapeutically effective amount” is the amount of gemcabene clinically proven to affect a significant reduction in inflammation. In some aspects, a therapeutically effective amount can be between 50 mg to 900 mg per subject.

The terms “effective” and “effectiveness” with regard to a treatment include both pharmacological effectiveness and physiological safety. Pharmacological effectiveness refers to the ability of the drug to reduce inflammation in the patient. Physiological safety refers to the level of toxicity, or other adverse physiological effects at the cellular, organ and/or organism level (adverse effects) resulting from administration of the drug.

The term “human coronavirus” as used herein refers to a positive-stranded RNA virus that has a lipid envelope studded with club-shaped trimeric s-glycoprotein projections that infect humans, and mutated strains thereof. Sexton et al., Journal of Virology 90:7415-7428 (2016).

The terms “HCoV-229E,” “HCoV-OC43,” “HCoV-NL63,” “HCoV-HKU1,” “SARS-CoV,” “MERS-CoV,” and “SARS-CoV-2” as used herein refer to the coronavirus pathogens currently known to infect humans. Lim et al., Diseases 2016, 4, 26; doi:10.3390/diseases4030026; Lai et al., International Journal of Antimicrobial Agent 55:1-9 (2020). New strains of human coronavirus can result due to mutations and genetic drift, and can be more or less virulent and more or less infectious.

The term “severe acute respiratory syndrome” or “SARS” as used herein refers to the viral respiratory disease caused by SARS-CoV.

The term “Middle East Respiratory Syndrome” or “MERS” as used herein refers to the viral respiratory disease caused by MERS-CoV.

The term “Coronavirus Disease 2019” or “COVID-19” as used herein refers to the viral respiratory disease caused by SARS-CoV-2.

The term “treat” or “treatment” refers to both therapeutic treatment and prophylactic or preventative measures, wherein the object is to prevent or slow down (lessen) an undesired physiological change or disorder, such as inflammation. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

### II. Gemcabene

The present disclosure provides methods of treating inflammation using gemcabene, or a pharmaceutically acceptable salt thereof. Gemcabene has been previously described, e.g., in U.S. Pat. No. 5,648,387, which is hereby incorporated by reference in its entirety. The compound is also known as 6-(5-carboxy-5-methylhexyloxy)-2,2-dimethyl-hexanoic acid and is represented by the structure shown below:

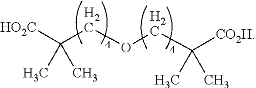

In some aspects, the present disclosure provides methods of treating inflammation using a pharmaceutically acceptable salt of gemcabene. In some aspects, the pharmaceutically acceptable salt can comprise the monocalcium salt of gemcabene, represented by the structure shown below:

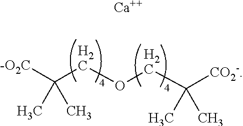

Various gemcabene calcium salt hydrates have been previously described, e.g., in U.S. Pat. No. 6,861,555, which is hereby incorporated by reference in its entirety.

Gemcabene can also be in the form of other pharmaceutically acceptable salts. In some aspects, such salts can be derived from inorganic or organic acids or bases.

Examples of suitable acid addition salts include acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate.

Examples of suitable base addition salts include ammonium salts; alkali metal salts, such as sodium and potassium salts; alkaline earth metal salts, such as calcium and magnesium salts; salts with organic bases, such as dicyclohexylamine salts, N-methyl-D-glucamine; and salts with amino acids such as arginine, lysine, and the like.

For example, Berge lists the following FDA-approved commercially marketed salts: anions acetate, besylate (benzenesulfonate), benzoate, bicarbonate, bitartrate, bromide, calcium edetate (ethylenediaminetetraacetate), camsylate (camphorsulfonate), carbonate, chloride, citrate, dihydrochloride, edetate (ethylenediaminetetraacetate), edisylate (1,2-ethanedisulfonate), estolate (lauryl sulfate), esylate (ethanesulfonate), fumarate, gluceptate (glucoheptonate), gluconate, glutamate, glycollylarsanilate (glycollamidophenylarsonate), hexylresorcinate, hydrabamine (N,N′-di(dehydroabietyl)-ethylenediamine), hydrobromide, hydrochloride, hydroxynaphthoate, iodide, isethionate (2-hydroxyethanesulfonate), lactate, lactobionate, malate, maleate, mandelate, mesylate (methanesulfonate), methylbromide, methylnitrate, methylsulfate, mucate, napsylate (2-naphthalenesulfonate), nitrate, pamoate (embonate), pantothenate, phosphate/diphosphate, polygalacturonate, salicylate, stearate, subacetate, succinate, sulfate, tannate, tartrate, teoclate (8-chlorotheophyllinate) and triethiodide; organic cations benzathine (N,N′-dibenzylethylenediamine), chloroprocaine, choline, diethanolamine, ethylenediamine, meglumine (N-methylglucamine) and procaine; and metallic cations aluminum, calcium, lithium, magnesium, potassium, sodium and zinc.

Berge additionally lists the following non-FDA-approved commercially marketed (outside the United States) salts: anions adipate, alginate, aminosalicylate, anhydromethylenecitrate, arecoline, aspartate, bisulfate, butylbromide, camphorate, digluconate, dihydrobromide, disuccinate, glycerophosphate, hemisulfate, hydrofluoride, hydroiodide, methylenebis(salicylate), napadisylate (1,5-naphthalenedisulfonate), oxalate, pectinate, persulfate, phenylethylbarbiturate, picrate, propionate, thiocyanate, tosylate and undecanoate; organic cations benethamine (N-benzylphenethylamine), clemizole (1-p-chlorobenzyl-2-pyrrolildine-1′-ylmethylbenzimidazole), diethylamine, piperazine and tromethamine (tris(hydroxymethyl)aminomethane); and metallic cations barium and bismuth.

### III. Compositions and Methods of Using

Pharmaceutical compositions comprising gemcabene can also comprise suitable carriers, excipients, and auxiliaries that may differ depending on the mode of administration.

In some aspects, the pharmaceutically acceptable carrier or vehicle, comprises a binder, filler, diluent, disintegrant, wetting agent, lubricant, glidant, coloring agent, dye-migration inhibitor, sweetening agent, flavoring agent, or a combination thereof.

Binders or granulators impart cohesiveness to a tablet to ensure the tablet remaining intact after compression. Suitable binders or granulators include, but are not limited to, starches, such as com starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Pan war gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof.

Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. In some aspects, the binder can comprise hydroxypropyl cellulose.

The binder or filler can be present from about 2% to about 49% by weight of the compositions of the disclosure provided herein or any range within these values. In some aspects, the binder or filler can be present in the composition of the disclosure from about 5% to about 15% by weight. In some aspects, the binder or filler can be present in the composition of the disclosure at about 5%, 6%, 7%, 8%, 9%, 8%, 10%, 11%, 12%, 13%, 14%, or 15% by weight or any range within any of these values.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets. In some aspects, the diluent can comprise lactose monohydrate. In another aspect, the diluent can comprise lactose monohydrate Fast-Flo 316 NF.

The compositions of the disclosure can comprise from about 5% to about 49% of a diluent by weight of composition or any range between any of these values. In some aspects, the diluent can be present in the compositions of the disclosure from about 15% to about 30% by weight. In some aspects, the diluent can be present in the composition of the disclosure at about 15%, 16%, 17%, 18%, 19%, 18%, 20%, 21%, 22%, 23%, 24%, 25%, 26%, 27%, 28%, 29%, or 30% by weight or any range within any of these values.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as com starch, potato starch, tapioca starch, and pre-gelatinized starch; clays; aligns; and mixtures thereof. The amount of disintegrant in the compositions of the disclosure can vary. In some aspects, the disintegrant can comprise croscarmellose sodium In some aspects, the disintegrant can comprise croscarmellose sodium NF (Ac-Di-Sol).

The compositions of the disclosure can comprise from about 0.5% to about 15% or from about 1% to about 10% by weight of a disintegrant. In some aspects, the compositions of the disclosure can comprise a disintegrant in an amount of about 5%, 6%, 7%, 8%, 9%, 8%, 10%, 11%, 12%, 13%, 14%, or 15% by weight of the composition or in any range within any of these values.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, com oil, and soybean oil; zinc stearate; ethyl oleate; ethyl laureate; agar; starch; lycopodium; silica or silica gels, such as AEROSIL® 200 (W.R. Grace Co., Baltimore, Md. and CAB-O-SIL® (Cabot Co. of Boston, Mass.); and mixtures thereof. In some aspects, the lubricant can comprise magnesium stearate.

The compositions of the disclosure can comprise about 0.1 to about 5% by weight of a lubricant. In some aspects, the compositions of the disclosure comprise a lubricant in an amount of about 0.5%, 0.6%, 0.7%, 0.8%, 0.9%, 0.8%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, or 3.0%, by weight of the composition or in any range within any of these values.

Suitable glidants include, but are not limited to, colloidal silicon dioxide, CAB-O-SIL® (Cabot Co. of Boston, Mass.), talc, including asbestos-free talc, and mixtures thereof.

Coloring agents include, but are not limited to, any of the approved, certified, water soluble FD&C dyes, and water insoluble FD&C dyes suspended on alumina hydrate, and color lakes, and mixtures thereof.

Flavoring agents include, but are not limited to, natural flavors extracted from plants, such as fruits, and synthetic blends of compounds that provide a pleasant taste sensation, such as peppermint and methyl salicylate, and mixtures thereof.

Sweetening agents include, but are not limited to, sucrose, lactose, mannitol, syrups, glycerin, sucralose; artificial sweeteners, such as saccharin and aspartame; and mixtures thereof.

Suitable emulsifying agents include, but are not limited to, gelatin, acacia, tragacanth, bentonite; surfactants, such as polyoxyethylene sorbitan monooleate (TWEEN® 20), polyoxyethylene sorbitan monooleate 80 (TWEEN® 80), and triethanolamine oleate; and mixtures thereof. Suspending and dispersing agents include, but are not limited to, sodium carboxymethylcellulose, pectin, tragacanth, Veegum, acacia, sodium carbomethylcellulose, hydroxypropyl methylcellulose, polyvinylpyrolidone, and mixtures thereof. Preservatives include, but are not limited to, glycerin, methyl and propylparaben, benzoic add, sodium benzoate, alcohol, and mixtures thereof. Wetting agents include, but are not limited to, propylene glycol monostearate, sorbitan monooleate, diethylene glycol monolaurate, polyoxyethylene lauryl ether, and mixtures thereof.

Solvents include, but are not limited to, glycerin, sorbitol, ethyl alcohol, syrup, and mixtures thereof.

Examples of non-aqueous liquids utilized in emulsions include, but are not limited to, mineral oil, cottonseed oil, and mixtures thereof. Organic acids include, but are not limited to, citric acid, tartaric acid, and mixtures thereof. Sources of carbon dioxide include, but are not limited to, sodium bicarbonate, sodium carbonate, and combinations thereof.

It should be understood that many carriers and excipients can serve several functions, even within the same formulation.

The compounds of the disclosure and the compositions of the disclosure can be formulated for administration by a variety of means including orally, parenterally, by inhalation spray, topically, or rectally in formulations containing pharmaceutically acceptable carriers, adjuvants and vehicles. The term “parenteral” as used here includes subcutaneous, intravenous, intramuscular, and intraarterial injections with a variety of infusion techniques. Intraarterial and intravenous injection as used herein includes administration through catheters.

The compounds of the disclosure and the compositions of the disclosure can be formulated in accordance with the routine procedures adapted for desired administration route. Accordingly, the compositions of the disclosure can take such forms as suspensions, solutions or emulsions in oily or aqueous vehicles, and can contain formulatory agents such as suspending, stabilizing and/or dispersing agents. The compounds of the disclosure and the compositions of the disclosure can be formulated as a preparation suitable for implantation or injection. Thus, for example, pharmaceutically acceptable salt of gemcabene and the compositions of the disclosure can be formulated with suitable polymeric or hydrophobic materials (e.g., as an emulsion in an acceptable oil) or ion exchange resins, or as sparingly soluble derivatives (e.g., as a sparingly soluble salt). The compounds of the disclosure and the compositions of the disclosure can be in powder form for constitution with a suitable vehicle, e.g., sterile pyrogen-free water, before use. Suitable formulations for each of these methods of administration can be found, for example, in Remington: The Science and Practice of Pharmacy, A Gennaro, ed., 20th edition, Lippincott, Williams & Wilkins, Philadelphia, Pa.

In some aspects, the compositions of the disclosure are suitable for oral administration. These compositions can comprise solid, semisolid, gelmatrix or liquid dosage forms suitable for oral administration. As used herein, oral administration includes buccal, lingual, and sublingual administration. Suitable oral dosage forms include, without limitation, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, syrups or any combination thereof. In some aspects, compositions of the disclosure suitable for oral administration are in the form of a tablet or a capsule. In some aspects, the composition of the disclosure can be in the form of a tablet. In some aspects, the composition of the disclosure can be in the form of a capsule. In some aspects, the compound of the disclosure can be in the form of a capsule.

In some aspects, capsules can be immediate release capsules. A non-limiting example of a capsule is a CONI-SNAP® hard gelatin capsule.

The compositions of the disclosure can be in the form of compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which can be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. A film coating can impart the same general characteristics as a sugar coating. Multiple compressed tablets are compressed tablets made by more than one compression cycle, including layered tablets, and press-coated or dry-coated tablets.

In some aspects, the coating can comprise a film coating. In some aspects, the film coating can comprise Opadry White and simethicone emulsion 30% USP.

In some aspects, the compound of the disclosure can be in the form of a tablet. In some aspects, the compound of the disclosure can be in the form of a compressed tablet. In some aspects, the compound of the disclosure can be in the form of a film-coated compressed tablet. In some aspects, the compositions of the disclosure can be in the form of film-coated compressed tablets.

In some aspects, the compositions of the disclosure can be prepared by fluid bed granulation of the compound of the disclosure with one or more pharmaceutically acceptable carriers, vehicles, and/or excipients. In some aspects, the compositions of the disclosure can be prepared by fluid bed granulation process and can provide a tablet formulation with good flowability, good compressibility, fast dissolution, good stability, and/or minimal to no cracking. In some aspects, the fluid bed granulation process can allow preparation of formulations having high drug loading, such as over 70% or over 75% of a compound of the disclosure.

The compositions of the disclosure can be in the form of soft or hard capsules, which can be made from gelatin, methylcellulose, starch, and/or calcium alginate. The hard gelatin capsule, also known as the dry-filled capsule (DFC), can comprise two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. In some aspects, soft gelatin shells can contain a preservative to prevent the growth of microorganisms. Suitable preservatives include, but are not limited to, those as described herein, including methyl- and propyl-parabens, sorbic acid, and combinations thereof. The liquid, semisolid, and solid dosage forms provided herein can be encapsulated in a capsule. Suitable liquid and semisolid dosage forms include, but are not limited to, solutions and suspensions in propylene carbonate, vegetable oils, triglycerides, and combinations thereof. Capsules containing such solutions can be prepared as described in U.S. Pat. Nos. 4,328,245; 4,409,239; and 4,410,545. The capsules can also be coated as known by those of skill in the art in order to modify or sustain dissolution of the active ingredient.

The compositions of the disclosure can be in liquid or semisolid dosage forms, including emulsions, solutions, suspensions, elixirs, and syrups. In some aspects, the emulsion can be a two-phase system, in which one liquid is dispersed in the form of small globules throughout another liquid, which can be oil-in-water or water-in-oil. Emulsions can include a pharmaceutically acceptable non-aqueous liquids or solvent, emulsifying agent, and preservative. Suspensions can include a pharmaceutically acceptable suspending agent and preservative. Aqueous alcoholic solutions can include a pharmaceutically acceptable acetal, such as a di-(lower alkyl)acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs can be clear, sweetened, and hydroalcoholic solutions. Syrups can be concentrated aqueous solutions of a sugar, for example, sucrose, and can comprise a preservative. For a liquid dosage form, for example, a solution in a polyethylene glycol can be diluted with a sufficient quantity of a pharmaceutically acceptable liquid carrier, e.g., water, to be measured conveniently for administration.

The compositions of the disclosure for oral administration can be also provided in the forms of liposomes, micelles, microspheres, or nanosystems. Miccellar dosage forms can be prepared as described in U.S. Pat. No. 6,350,458.

The compositions of the disclosure can be provided as non- effervescent or effervescent, granules and powders, to be reconstituted into a liquid dosage form. Pharmaceutically acceptable carriers and excipients used in the non-effervescent granules or powders can include, but are not limited to, diluents, sweeteners, wetting agents, and mixtures thereof. Pharmaceutically acceptable carriers and excipients used in the effervescent granules or powders can include, but are not limited to, organic acids, a source of carbon dioxide, and mixtures thereof.

Coloring and flavoring agents can be used in all of the above dosage forms. In addition, flavoring and sweetening agents can be especially useful in the formation of chewable tablets and lozenges.

The compositions of the disclosure can be formulated as immediate or modified release dosage forms, including delayed-, extended, pulsed-, controlled, targeted-, and programmed-release forms.

The compositions of the disclosure can comprise another active ingredient that does not impair the composition's therapeutic or prophylactic efficacy and/or can comprise a substance that augments or supplements the composition's efficacy.

The tablet dosage forms can comprise a pharmaceutically acceptable salt of gemcabene in powdered, crystalline, or granular form, and can further comprise one or more carriers and/or one or more vehicles described herein, including binders, disintegrants, controlled-release polymers, lubricants, diluents, and/or colorants.

In some aspects, the compositions of the disclosure can comprise from about 50 mg to about 900 mg, about 150 mg to about 600 mg, or about 150 mg to about 300 mg of a compound of the disclosure. In some aspects, the compositions of the disclosure can comprise a compound of the disclosure in an amount of about 25 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, or an amount ranging from and to any of these values. In some aspects, the compositions of the disclosure can comprise about 50 mg of a compound of the disclosure. In some aspects, the compositions of the disclosure can comprise about 150 mg of a compound of the disclosure. In some aspects, the compositions of the disclosure can comprise about 300 mg of a compound of the disclosure. In some aspects, the compositions of the disclosure can comprise about 600 mg of a compound of the disclosure.

In some aspects, the compositions of the disclosure can comprise a compound of the disclosure in an amount that is a molar equivalent to about 25 mg to about 900 mg, about 150 mg to about 600 mg, or about 150 mg to about 300 mg of gemcabene, or a pharmaceutically acceptable salt thereof. In some aspects, the compositions of the disclosure can comprise a compound of the disclosure in an amount that is a molar equivalent to about 25 mg, about 30 mg, about 40 mg, 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, or about 900 mg gemcabene or a pharmaceutically acceptable salt thereof, or an amount ranging from and to any of these values. In some aspects, the compositions of the disclosure can comprise a pharmaceutically acceptable salt of gemcabene or a pharmaceutically acceptable salt thereof, in an amount that is a molar equivalent to about 50 mg. In some aspects, the compositions of the disclosure can comprise a compound of the disclosure in an amount that is a molar equivalent to about 150 mg of he disclosure in an amount that is a molar equivalent to about 300 mg. In some aspects, the compositions of the disclosure can comprise a compound of the disclosure in an amount that is a molar equivalent to about 600 mg.

In other aspects, the compositions of the disclosure can comprise a compound of the disclosure in an amount of about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, or any amount ranging from and to these values.

In certain aspects, the compositions of the present disclosure can be administered from 1 to 10 times a day. In some aspects, the compositions of the present disclosure can be administered from 1 to 5 times a day. In some aspects, the compositions of the present disclosure can be administered 1, 2, 3, 4, 5, 6, 7, 8, 9, or 10 times a day. In some aspects, the compositions of the present disclosure can be administered once a day. In some aspects, the compositions of the present disclosure can be administered twice a day.

In certain aspects, the subject that can be treated with the compounds and the compositions of the present disclosure is a nonhuman mammal. In some aspects, the subject that can be treated is a human.

In certain aspects, the compounds and compositions described herein can be used to treat and/or prevent a cytokine storm. In some aspects, the cytokine storm can be caused or derived from a viral infection, a monocolonal antibody, a bacterial infection, a protozoan infection, a fungal infection, a prion, an animal bite, a insect bite or sting, exposure to a toxin, a snake bite, a spider bite, a scorpion sting, sea creature bites, spines, and stings (such as from a sea urchin, a jelly fish, stonefish, blue-ringed octopus, or sea snake) stinging plants (such as nettles and gympie-gympie), tetnus toxin, chemical agents, physical agents (such as heat, cold, radiation or physical trauma), a gene therapeutic agent, a vaccine, a vaccine booster, an allergan, or a chimeric antigen receptor therapeutic agent (such as trisagenlecleucel), or combinations thereof.

In certain aspects the cytokine storm can be caused by a monoclonal antibody. Examples of therapeutic monocolonal antibodies that have been reported to induce cytokine storms include, but are not limited to, rituximab, muromonab-CD3, alemtuzumab, theralizumab, and combinations thereof.

In certain aspects the cytokine storm can caused by a viral infection. In certain aspects the viral infection can be caused by an RNA virus or a DNA virus. Examples of RNA viruses include, but are not limited to, influenza (including, but not limited to, influenza A, influenza B and its subtypes), SARS, coronavirus, HTLV-1, and HIV, as well as mutations thereof. Examples of DNA viruses include, but are not limited to, HPV, CMV, BKV, HSV, VZV, EBV, as well as mutations thereof. In some aspects, the virus has a filamentous, isometric, enveloped, or a head and tail conformation.

In some aspects, the viral infection is caused by a DNA virus, e.g., hepatitis B virus, hepatitis C virus, human cytomegalovirus, or herpes simplex virus type 1. In some aspects, the DNA virus is hepatitis C virus.

In some aspects, the viral infection is caused by a RNA virus, e.g., respiratory syncytial virus, parainfluenza-3 virus, bovine viral diarrhea virus, Venezuelan equine encephalomyelitis virus, Dengue virus, yellow fever virus, Coxsackie B3 virus, encephalomyocarditis virus, influenza A virus, Zika virus, Ebola virus, Junin virus, Lassa Fever virus, Chikungunya virus, or coronavirus. In some aspects, the RNA virus is a coronavirus. In some aspects, the coronavirus is a human coronavirus, or a mutated strain thereof, or an animal coronavirus, or a mutated strain thereof. In some aspects, the coronavirus is a human coronavirus, or a mutated strain thereof.

In some aspects, the coronavirus is a human coronavirus. In some aspects, the coronavirus is a mutated strain of a human coronavirus. In some aspects, the coronavirus is an animal coronavirus, or a mutated strain thereof. In some aspects, the coronavirus is an animal coronavirus. In some aspects, the coronavirus is a mutated strain of an animal coronavirus.

In some aspects, the human coronavirus is HCoV-229E, HCoV-OC43, HCoV-NL63, HCoV-HKU1, SARS-CoV, MERS-CoV, or SARS-CoV-2, or a mutated strain of HCoV-229E, HCoV-OC43, HCoV-NL63, HCoV-HKU1, SARS-CoV, MERS-CoV, or SARS-CoV-2. In some aspects, the human coronavirus is HCoV-229E, HCoV-OC43, HCoV-NL63, HCoV-HKU1, SARS-CoV, MERS-CoV, or SARS-CoV-2. In some aspects, the human coronavirus is a mutated strain of HCoV-229E, HCoV-OC43, HCoV-NL63, HCoV-HKU1, SARS-CoV, MERS-CoV, or SARS-CoV-2. In some aspects, the human coronavirus is HCoV-229E. In some aspects, the human coronavirus is a mutated strain of HCoV-229E. In some aspects, the human coronavirus is HCoV-OC43. In some aspects, the human coronavirus is a mutated strain of HCoV-OC43. In some aspects, the human coronavirus is HCoV-NL63.

In some aspects, the human coronavirus is a mutated strain of HCoV-NL63. In some aspects, the human coronavirus is HCoV-HKU1. In some aspects, the human coronavirus is a mutated strain of HCoV-HKU1. In some aspects, the human coronavirus is SARS-CoV. In some aspects, the human coronavirus is a mutated strain of SARS-CoV. In some aspects, the human coronavirus is MERS-CoV. In some aspects, the human coronavirus is a mutated strain of MERS-CoV. In some aspects, the human coronavirus is SARS-CoV-2. In some aspects, the human coronavirus is a mutated strain of SARS-CoV-2.

In some aspects, the infectious disease is the common cold, pneumonia, bronchitis, SARS, MERS, or COVID-19. In some aspects, the infectious disease is the common cold. In some aspects, the infectious disease is pneumonia. In some aspects, the infectious disease is bronchitis. In some aspects, the symptom of an infectious caused by a human coronavirus is fever, cough, shortness of breath, pneumonia, inflammation, and/or cytokine storm.

In some aspects, the virus can be a coronavirus selected from is HCoV 229E, HCoV OC43, HCoV NL63, HCoV HKU1, SARS CoV, MERS CoV, and SARS CoV-2, a mutated strain thereof, and any combination thereof. In some aspects, the virus can be a coronavirus which can be SARS CoV-2 or a mutated strain of SARS CoV-2.

In certain aspects, the viral infection can be a gastrointestinal tract infection, a respiratory infection, or a combination thereof. In certain aspects, the viral infection can be a respiratory infection that causes an inflammatory lung condition. In certain aspects, the inflammatory lung condition can be an acute and/or comprises an interstitial lung disease. In certain aspects, the inflammatory lung condition can be hypercytokinaemia, haemophagocytic lymphohistiocytosis, pneumonia, acute respiratory distress syndrome, or systemic inflammatory response syndrome.

The compounds and compositions described herein can also be used for treating multisystem inflammatory syndrome. In some aspects, the multisystem inflammatory system can comprise fever that lasts more than 5 days and gets higher, severe abdominal pain, vomiting, or diarrhea, bloodshot eyes, skin rash, change in skin color, which can include becoming pale, patchy, or blue, difficulty feeding or too sick to drink, trouble breathing or quick breathing, chest pain or racing heart, confusion, irritability, or lethargy, or any combination thereof. In some aspects, the multisystem inflammatory syndrome can further comprise swelling and redness in the hands and feet, redness or cracking in the lips or tongue, swollen lymph nodes in the neck, or any combination thereof.

In certain aspects, the subject can have a condition that makes him/her more likely to experiencee a cytokine storm. In certain aspects, the condition can comprise a metabolic disease. In some aspects, the subject can have type 1, type 2 and/or gestational diabetes, familial hypercholesterolemia, elevated triglycerides, above normal BMI, a BMI >40, cystic fibrosis, a history of smoking, idiopathic pulmonary fibrosis, idiopathic respiratory distress syndrome, non-alcoholic fatty liver disease (NAFLD), Non-Alcoholic Steatohepatitis (NASH), liver disease, cirrhosis, hypertension, familial partial lipodystrophy, compromised respiratory function, asthma, chronic lung disease, chronic obstructive pulmonary disease (COPD), emphysema, chronic bronchitis, chronic kidney disease, one or more hemoglobin disorders, sickle cell anemia, thalassemia, anemia, a compromised immune system due to cancer treatment, an autoimmune disease, an age over 65, cardiovascular disease, a heart condition, heart failure, coronary artery disease, congenital heart disease, cardiomyopathies, and pulmonary hypertension, vitamin D deficiency, or any combination thereof. In some aspects, a subject who has a condition that makes him/her more likely to experiencee a cytokine storm can be treated prophylactically with the compounds and/or compositions of the present disclosure.

In certain aspects, the subject can be less than 12 years old, less than 18 years old, less than 30 years old, less than 50 years old, less than 60 years old, less than 75 years old, less than 80 years old, less than 85 years old, less than 90 years old, less than 95 years old, or less than 100 years old. In some aspects, the subject can be pediatric. In some aspects, the subject can be over the age of 50, 55, 60, 65, 70, 75, 80, 85, or 90 years old.

In some aspects, the compounds and compositions of the disclosure can be used in combination with one or more additional therapeutic agents. Accordingly, in certain aspects, a method of treating a cytokine storm disclosed herein comprises administering gemcabene or a pharmaceutically acceptable salt thereof in combination with one or more additional therapeutic agents. In some aspects, gemcabene or a pharmaceutically acceptable salt thereof can be used in combination with one or more antiviral agents, such that multiple aspects of the inflammatory response can be targeted. Non-limiting examples of antiviral agents that can be used in the present methods include acyclovir, famcyclovir, ganciclovir, idoxuridine, foscamet, fomivirsen, pencyclovir, trifluridine, tromantadine, valacyclovir, valgancyclovir, vidarbine, cidofavir, docosanol, amantadine, oseltamivir, peramivir, rimantadine, zanamivir, fomivirsen, enfuvirtide, imiquimod, interferon, ribavirin, viramidine, ziduvidine, didanosine, stauvidine, zalcitabine, lamivudine, abacavir, tenofovir, nevirapine, efavirenz, delavirdine, saquinavir, indinavir, atazanavir, ritonavir, nelfinavir, amprenavir, lopinavir, tipranavir, and combinations thereof.

In some aspects, gemcabene or a pharmaceutically acceptable salt thereof can be used in combination with one or more anti-inflammatory agents, such that multiple aspects of the inflammatory response can be targeted. Non-limiting examples of anti-inflammatory agents that can be used in the present methods include aspirin, sodium salycilate, fenoprofen, diflunixal, ibuprofen, ketoprofen, naproxen, flurbiprofen, diclofenac, ketorolac, tolmetin, etodolac, indomethacin, sulindac, aceclofenac, mefenamic acid, meclofenamic acid, tolfenamic acid, piroxicam, phenylbutazone, nabumetone, tenoxicam, indomethacin, sulindac, minisulide, nimesulide, meloxicam, etoricoxib, valdecoxib, parecoxib, paracetamol, nefopam, cortisol, cortisone, hydrocortisone, dexamethasone, fludrocortisone, methylprednisolone, prednisolone, and combinations thereof.

In some aspects, the compounds and compositions of the disclosure can be administered to the subject prior to or after the administration of the additional therapeutic agent. In other aspects, the compounds and compositions can be administered to the subject concurrently with the additional therapeutic agent. In certain aspects, compounds and compositions of the disclosure and the additional therapeutic agent can be administered concurrently as a single composition in a pharmaceutically acceptable carrier. In other aspects, the compounds and compositions of the disclosure and the additional therapeutic agent are administered concurrently as separate compositions.

In certain aspects, the compounds and compositions of the disclosure can be administered for a period of from about 7 days to 6 months. In some aspects, the compounds and compositions of the disclosure can be administered for a period of from about 7 days to about 3 months. In some aspects, the compounds and compositions of the disclosure can be administered for a period of from about 7 days to about 2 months. In some aspects, the compounds and compositions of the disclosure can be administered for a period of from about 7 days to about 45 days. In some aspects, the compounds and compositions of the disclosure can be administered for about 7 to about 28 days. In some aspects, the compounds and compositions of the disclosure can be administered for about 7 to about 14 days. In certain aspects, the compounds and compositions of the disclosure can be administered for about 7, about 8, about 9, about 10, about 11, about 12, about 13, about 14, about 15, about 16, about 17, about 18, about 19, about 20, about 21, about 22, about 23, about 24, about 25, about 26, about 27, or about 28 days.

In certain aspects, the compounds and compositions of the disclosure can be administered once a day for a period of from about 7 days to 6 months. In some aspects, the compounds and compositions of the disclosure can be administered once a day for a period of from about 7 days to 3 months. In some aspects, the compounds and compositions of the disclosure can be administered for a period of from about 7 days to about 2 months. In some aspects, the compounds and compositions of the disclosure can be administered for a period of from about 7 days to about 45 days. In some aspects, the compounds and compositions of the disclosure can be administered once a day for about 7 to about 28 days. In some aspects, the compounds and compositions of the disclosure can be administered once a day for about 7 to about 14 days. In certain aspects, the compounds and compositions of the disclosure can be administered once a day for about 7, about 8, about 9, about 10, about 11, about 12, about 13, about 14, about 15, about 16, about 17, about 18, about 19, about 20, about 21, about 22, about 23, about 24, about 25, about 26, about 27, or about 28 days.

In certain aspects, the compounds and compositions of the disclosure can be administered twice a day for a period of from about 7 days to 6 months. In some aspects, the compounds and compositions of the disclosure can be administered twice a day for a period of from about 7 days to 3 months. In some aspects, the compounds and compositions of the disclosure can be administered for a period of from about 7 days to about 2 months. In some aspects, the compounds and compositions of the disclosure can be administered for a period of from about 7 days to about 45 days. In some aspects, the compounds and compositions of the disclosure can be administered twice a day for about 7 to about 28 days. In some aspects, the compounds and compositions of the disclosure can be administered twice a day for about 7 to about 14 days. In certain aspects, the compounds and compositions of the disclosure can be administered twice a day for about 7, about 8, about 9, about 10, about 11, about 12, about 13, about 14, about 15, about 16, about 17, about 18, about 19, about 20, about 21, about 22, about 23, about 24, about 25, about 26, about 27, or about 28 days.

In some aspects, the subject can be treated for multiple cytokine storms over a period of time. For example, in some aspects the subject can experience a first cytokine storm after receiving a vaccine and can experience a second cytokine storm after receiving a booster for that vaccine. In some aspects, the subject can experience a first cytokine storm in response to a viral infection and can experience a second cytokine storm in response to a subsequent viral infection after being treated for the first cytokine storm.

In certain aspects, subjects who are treated with the compounds and/or compositions of the present disclosure experience a shorter recovery time than those receiving other treatemnts or no treatment. In some aspects, subjects who are treated with the compounds and/or compositions of the present disclosure experience a higher survival rate than those receiving other treatments or no treatment. In some aspects, subjects who are treated with the compounds and/or compositions of the present disclosure are less likely to require respirator therapy than those receiving other treatments or no treatment. In some aspects, subjects who are treated with the compounds and/or compositions of the present disclosure experience fewer secondary complications resulting from the cytokine storm than those receiving other treatments or no treatment.

### EXAMPLES

### Example 1: Prophylactic Treatment of High-Risk Covid-19 Patients with Gemcabene

Subjects diagnosed with Covid-19 who meet high risk criteria are administered 300 mg-900 mg/day of gemcabene alone or in combination with other therapeutic agents for a period of 7 to 28 days to reduce the risk of progessing to respiratory failure. Subjects are monitored for improvement in symptoms.

### Example 2: Prophylactic Treatment of Patients Suffering Viral-Induced Cytokine Storm with Gemcabene

Subjects diagnosed with a viral infection who meet high risk criteria are administered 300 mg-900 mg/day of gemcabene alone or in combination with other therapeutic agents for a period of 7 to 28 days to reduce the risk of progessing to respiratory failure. Subjects are monitored for improvement in symptoms.

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

What is claimed is:

1. A method of treating and/or preventing a cytokine storm in a subject in need thereof, the method comprising administering to the subject a pharmaceutical composition comprising an effective amount of gemcabene, or a pharmaceutically acceptable salt thereof.

2. The method of claim 1, wherein the cytokine storm is caused by or derived from a viral infection, a bacterial infection, a protozoan infection, a fungal infection, a prion, a vaccine, a vaccine booster, a gene therapeutic agent, a monoclonal antibody, or an allergan, or a combination thereof.

3. The method of claim 2, wherein the cytokine storm is caused by or derived from a viral infection.

4. The method of claim 3, wherein the viral infection is a respiratory or gastrointestinal tract infection, or a combination thereof.

5. The method of claim 3 or 4, wherein the viral infection is caused by an RNA or DNA virus.

6. The method of claim 4 or 5, wherein the virus has a filamentous, isometric, enveloped, or a head and tail conformation.

7. The method of claim 5, wherein the virus is a coronavirus.

8. The method of claim 7, wherein the coronavirus is HCoV 229E, HCoV OC43, HCoV NL63, HCoV HKU1, SARS CoV, MERS CoV, SARS CoV-2, or a mutated strain thereof, or any combination thereof.

9. The method of claim 8, wherein the coronavirus is SARS CoV-2.

10. The method of claim 8, wherein the coronavirus is a mutated strain of SARS CoV-2.

11. The method of any one of claims 1 to 10, wherein the effective amount of gemcabene, or the pharmaceutically acceptable salt thereof, is from about 25 mg/day to about 900 mg/day.

12. The method of any one of claims 1 to 11, wherein the effective amount of gemcabene, or the pharmaceutically acceptable salt thereof, is about 25 mg, about 50 mg, about 75 mg, about 100 mg, about 150 mg, about 200 mg, about 300 mg, about 400 mg, about 450 mg, about 500 mg, about 600 mg, about 700 mg, about 750 mg, about 800 mg, or about 900 mg.

13. The method of any one of claims 1 to 12, wherein the gemcabene, or the pharmaceutically acceptable salt thereof, is administered for about 7 to about 28 days.

14. The method of any one of claims 1 to 13, wherein the gemcabene, or the pharmaceutically acceptable salt thereof, is administered for about 7 to about 14 days.

15. The method of any one of claims 1 to 13, wherein the gemcabene, or the pharmaceutically acceptable salt thereof, is administered for about 7, about 8, about 9, about 10, about 11, about 12, about 13, about 14, about 15, about 16, about 17, about 18, about 19, about 20, about 21, about 22, about 23, about 24, about 25, about 26, about 27, or about 28 days.

16. The method of any one of claims 1 to wherein the pharmaceutical composition comprises the monocalcium salt of gemcabene.

17. A method of treating an inflammatory lung condition or disease caused by a viral infection in a subject in need thereof, the method comprising administering to the subject a pharmaceutical composition comprising an effective amount of gemcabene, or a pharmaceutically acceptable salt thereof.

18. The method of claim 17, wherein the inflammatory lung condition or disease comprises an acute inflammation of the lung and/or an interstitial lung disease.

19. The method of claim 17 or 18, wherein the viral infection comprises an infection by a coronavirus, an influenza virus, and/or HIV.

20. The method of claim 19, wherein the coronavirus is HCoV 229E, HCoV OC43, HCoV NL63, HCoV HKU1, SARS CoV, MERS CoV, SARS CoV-2, or a mutated strain thereof, or any combination thereof.

21. The method of claim 20, wherein the coronavirus is SARS CoV-2.

22. The method of claim 20, wherein the coronavirus is a mutated strain of SARS CoV-2.

23. The method of any one of claims 17 to 22, wherein the effective amount of gemcabene, or the pharmaceutically acceptable salt thereof, is from about 25 mg/day to about 900 mg/day.

24. The method of any one of claims 17 to 23, wherein the effective amount of gemcabene, or the pharmaceutically acceptable salt thereof, is about 25 mg/day, about 50 mg/day, about 75 mg/day, about 100 mg/day, about 150 mg/day, about 200 mg/day, about 300 mg/day, about 400 mg/day, about 450 mg/day, about 500 mg/day, about 600 mg/day, about 700 mg/day, about 750 mg, /day about 800 mg/day, or about 900 mg/day.

25. The method of any one of claims 17 to 24, wherein the gemcabene, or the pharmaceutically acceptable salt therefo, is administered for about 7 to about 28 days.

26. The method of any one of claims 17 to 25, wherein the gemcabene, or the pharmaceutically acceptable salt thereof, is administered for about 7 to about 14 days.

27. The method of any one of claims 17 to 25, wherein the gemcabene, or the pharmaceutically acceptable salt thereof, is administered for about 7, about 8, about 9, about 10, about 11, about 12, about 13, about 14, about 15, about 16, about 17, about 18, about 19, about 20, about 21, about 22, about 23, about 24, about 25, about 26, about 27, or about 28 days.

28. The method of any one of claims 17 to 27, wherein the pharmaceutical composition comprises the monocalcium salt of gemcabene.

29. The method of any one of claims 17 to 28, wherein the inflammatory lung condition or disease caused by the viral infection is hypercytokinaemia, haemophagocytic lymphohistiocytosis, pneumonia, acute respiratory distress syndrome, or systemic inflammatory response syndrome.

30. The method of any one of claims 17 to 29, wherein the inflammatory lung condition or disease caused by the viral infection is acute respiratory distress syndrome.

31. The method of any one of claims 1 to 30, wherein the subject has a metabolic disease.

32. The method of claim 31, wherein the subject has type 1 diabetes, type 2 diabetes, gestational diabetes, familial hypercholesterolemia, elevated triglycerides, above normal BMI, a BMI >40, cystic fibrosis, a history of smoking, idiopathic pulmonary fibrosis, idiopathic respiratory distress syndrome, non-alcoholic fatty liver disease (NAFLD), non-alcoholic steatohepatitis (NASH), liver disease, cirrhosis, hypertension, familial partial lipodystrophy, compromised respiratory function, asthma, chronic lung disease, chronic obstructive pulmonary disease (COPD), emphysema, chronic bronchitis, chronic kidney disease, a hemoglobin disorder, sickle cell anemia, thalassemia, anemia, a compromised immune system from cancer treatment, an autoimmune disease, an age of 65 or older, cardiovascular disease, a heart condition, heart failure, coronary artery disease, congenital heart disease, cardiomyopathies, pulmonary hypertension, vitamin D deficiency, or a combination thereof.

33. A method of treating a multisystem inflammatory syndrome in a subject in need thereof, the method comprising administering to the subject a pharmaceutical composition comprising an effective amount of gemcabene, or a pharmaceutically acceptable salt thereof.

34. The method of claim 33, wherein the syndrome comprises fever that lasts more than 5 days and gets higher, severe abdominal pain, vomiting, or diarrhea, bloodshot eyes, skin rash, change in skin color, which can include becoming pale, patchy, or blue, difficulty feeding or too sick to drink, trouble breathing or quick breathing, chest pain or racing heart, confusion, irritability, or lethargy, or any combination thereof.

35. The method of claim 34, wherein the syndrome further comprises swelling and redness in the hands and feet, redness or cracking in the lips or tongue, swollen lymph nodes in the neck, or any combination thereof.

36. The method of claim 33, wherein the syndrome is associated with a viral infection.

37. The method of claim 36, wherein the viral infection comprises an infection by a coronavirus, an influenza virus, and/or HIV.

38. The method of claim 37, wherein the coronavirus is HCoV 229E, HCoV OC43, HCoV NL63, HCoV HKU1, SARS CoV, MERS CoV, SARS CoV-2, or a mutated strain thereof, or any combination thereof.

39. The method of claim 38, wherein the coronavirus is SARS CoV-2.

40. The method of claim 38, wherein the coronavirus is a mutated strain of SARS CoV-2.

41. The method of any one of claims 34 to 40, wherein the effective amount of gemcabene, or the pharmaceutically acceptable salt thereof, is from about 25 mg/day to about 900 mg/day.

42. The method of any one of claims 34 to 41, wherein the effective amount of gemcabene, or the pharmaceutically acceptable salt thereof, is about 25 mg/day, about 50 mg/day, about 75 mg/day, about 100 mg/day, about 150 mg/day, about 200 mg/day, about 300 mg, /day about 400 mg/day, about 450 mg/day, about 500 mg/day, about 600 mg/day, about 700 mg/day, about 750 mg/day, about 800 mg/day, or about 900 mg/day.

43. The method of any one of claims 34 to 42, wherein the gemcabene, or the pharmaceutically acceeptable salt thereof, is administered for about 7 to about 28 days.

44. The method of any one of claims 34 to 43, wherein the gemcabene, or the pharmaceutically acceeptable salt thereof, is administered for about 7 to about 14 days.

45. The method of any one of claims 34 to 43, wherein the gemcabene, or the pharmaceutically acceeptable salt thereof, is administered for about 7, about 8, about 9, about 10, about 11, about 12, about 13, about 14, about 15, about 16, about 17, about 18, about 19, about 20, about 21, about 22, about 23, about 24, about 25, about 26, about 27, or about 28 days.

46. The method of any one of claims 34 to 45, wherein the pharmaceutical composition comprises gemcabene calcium.

47. The method of any one of claims 1 to 46, further comprising administering one or more optional therapeutic agents to the subject.

48. The method of any one of claims 1 to 47, wherein the subject is a pediatric.

49. The method of any one of claims 1 to 47, wherein the subject is less than 18 years old, less than 30 years old, less than 50 years old, less than 60 years old, less than 75 years old, less than 80 years old, less than 85 years old, less than 90 years old, less than 95 years old, and/or less than 100 years old.

50. A kit comprising gemcabene, or a pharmaceutically acceptable salt thereof, for administering gemcabene, or a pharmaceutically acceptable salt thereof, to a subject having an inflammatory lung condition or disease caused by a viral infection, acute inflammation of the lung, or idiopathic pulmonary fibrosis.
